(19) United States
(12) Patent Application Publication
(10) Pub. No.: US 2006/0293323 A1
(43) Pub. Date: Dec. 28, 2006

(54) Therapeutic Combinations of erb B Kinase Inhibitors and Antineoplastic Therapies

(76) Inventors: William Leon Elliott, Ann Arbor, MI (US); David William Fry, Ypsilanti, MI (US)

Correspondence Address: PFIZER INC, 150 EAST 42ND STREET, 5TH FLOOR - STOP 49, NEW YORK, NY, 10017-5612, US

(21) Appl. No.: 11/464,249

(22) Filed: Aug. 14, 2006

Related U.S. Application Data
(63) Continuation of application No. 10/632,281, filed on Aug. 1, 2003, now abandoned.
(60) Provisional application No. 60/462,247, filed on Apr. 11, 2003.
(60) Provisional application No. 60/401,705, filed on Aug. 7, 2002.

(51) Int. Cl. A61K 31/5377; A61K 31/7072; A61K 31/7048; A61K 31/704; A61K 31/337; A61K 31/282; A61K 31/4745

(52) U.S. Cl. 514/234.2; 424/649; 514/27; 514/34; 514/49; 514/449; 514/492; 514/283

(57) ABSTRACT

Administration of an irreversible tyrosine kinase inhibitor such as CI-1033 in combination with one or more other antineoplastic agent(s), or ionizing radiation is synergistic for treating cancer.

### FIELD OF THE INVENTION

The invention concerns a method for treating cell proliferative disorders utilizing an erbB receptor tyrosine kinase inhibitor in conjunction with conventional antineoplastic agents and modalities. The use of this combination of agents in a therapeutic protocol provides unexpectedly greater efficacy than employing the single agents alone.

### BACKGROUND OF THE INVENTION

Pathological conditions resulting in inappropriate proliferation of cells are a common cause of human disease. Benign mammalian disease differs from malignant disease (cancer) primarily by the inability to spread from one part of the body to another and their generally slower growth rate. Both can kill or otherwise disable its victim. Internal adhesions and scarring after abdominal surgery can lead to bowel strangulation and death. Blindness from diabetes mellitus results from the inappropriate growth of new blood vessels inside the eye. Benign neurofibromas cause disfigurement. Psoriasis, a skin disease, results from the inappropriate overgrowth of otherwise normal cells. Cancers are one of the leading causes of death. While cancer chemotherapy has advanced dramatically in recent years. Many tumors can be effectively treated utilizing compounds that are either naturally occurring products or synthetic agents. In addition, other cancer therapies, such as ionizing radiation are used effectively in the treatment of certain cancers. Cancer therapy often entails use of a combination of agents, generally as a means of providing greater therapeutic effects and reducing the toxic effects that are often encountered with the individual agents when used alone.

Chemotherapy is also a mainstay of cancer treatment and is routinely used with success against many types of cancer and other hyperproliferative cellular disorders. Nevertheless, certain types of cancer are not amenable to chemotherapy protocols that are currently in use. Some types of tumors simply do not respond to standard methods of chemotherapy, or respond for a time and later become insensitive, resulting in a recurrence of the cancer. New methods that enhance current chemotherapy protocols are highly desirable.

Many antineoplastic agents have been used therapeutically to treat cancers. Among the most widely used are gemcitabine, paclitaxel, docetaxel, carboplatin, cisplatin, topotecan, CPT-11 etoposide, doxorubicin, and capecitabine. Many of these agents have limited therapeutic effect. Most of these agents must be used at such high doses that severe side effects are common. In addition to the chemical agents noted above, radiation therapy has been employed successfully to halt disease progression or cause tumor regression.

Gemcitabine is the generic name assigned to 2′-deoxy-2′,2′-difluoro-cytidine. It is commercially available as the monohydrochloride salt, and as the β-isomer. It is also known chemically as 1-(4-amino-2-oxo-1H-pyrimidin-1-yl)-2-desoxy-2,2-difluororibose. Gemcitabine is disclosed in U.S. Pat. Nos. 4,808,614 and 5,464,826, which are incorporated herein by reference for their teaching of how to synthesize, formulate, and use gemcitabine for treating susceptible neoplasms. The commercial formulation of gemcitabine hydrochloride as a single agent is indicated as first-line treatment for patients with locally advanced (nonresectable Stage II or Stage III) or metastatic (Stage IV) adenocarcinoma of the pancreas or lung cell carcinoma (NSCLC), and is commonly used in patients previously treated with 5-fluorouracil. It also is routinely used in combination with other known antineoplastic agents, most notably with ionizing radiation. No synergistic combinations have, however, heretofore been reported.

Paclitaxel is a natural product mitotic inhibitor. It is an antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. In addition, paclitaxel induces abnormal arrays or bundles of microtubules throughout the cell cycle and multiple asters of microtubules during mitosis. Paclitaxel is indicated primarily for ovarian carcinoma and breast cancer, although it is useful in treating other cancers as well. Paclitaxel is disclosed in U.S. Pat. Nos. 5,496,804, 5,641,803, 5,670,537 and 6,510,398, which are incorporated herein by reference for their teaching of how to synthesize, formulate, and use paclitaxel for treating susceptible neoplasms. Use of paclitaxel is generally accompanied by undesirable side effects, including hypersensitivity reactions, hypotension, bradycardia, hypertension, nausea and vomiting, and injection site reactions. Paclitaxel is commercially available as Taxol® (Bristol-Myers Squibb).

Docetaxel is a semi-synthetic compound belonging to the taxoid family. It is an antimicrotubule agent that promotes the assembly of microtubules from tubulin dimers and stabilizes microtubules by preventing depolymerization, This stability results in the inhibition of the normal dynamic reorganization of the microtubule network that is essential for vital interphase and mitotic cellular functions. In addition, docetaxel induces abnormal arrays or bundles of microtubules throughout the cell cycle and multiple asters of microtubules during mitosis. Docetaxel is indicated primarily for breast cancer and cell lung cancer, although it is useful in treating other cancers as well. Docetaxel is disclosed in U.S. Pat. Nos. 4,814,470, 5,438,072, 5,698,582 and 5,714,512, which are incorporated herein by reference for their teaching of how to synthesize, formulate, and use docetaxel for treating susceptible neoplasms. Use of docetaxel is generally accompanied by undesirable side effects, including hypersensitivity reactions, hypotension, bradycardia, hypertension, nausea and vomiting, and injection site reactions. Docetaxel trihydrate is commercially available as Taxotere® (Aventis Phamaceutical Products, Inc).

Carboplatin and cisplatin are the generic names assigned to diammine [1,1-cyclobutane-dicarboxylato(2-)-0,0′]-, (SP-4-2) platinum and cis-diaminodichloroplatinum (II), respectively. Both are commercially available as preparations for IV injection. Carboplatinum is disclosed in U.S. Pat. No. 4,657,927, which is incorporated herein by reference for its teaching of how to synthesize, formulate, and use carboplatin for treating susceptible neoplasms. Similarly, cisplatin is disclosed in German patent DE 2,318,020, which are incorporated herein by reference for their teaching of how to synthesize, formulate, and use cisplatin for treating susceptible neoplasms. Carboplatin and cisplatin alkylate DNA and thus interfere with DNA replication and transcription. Carboplatin and cisplatin are used in the treatment of cancers of the testis, ovary, endometrium, cervix, bladder, head and neck, gasterointestinal tract, lung, soft tissue and bone sarcomas, and non-Hodgkins lymphoma. Use of platinum compounds is generally accompanied by several side effects including myelosuppression, nausea and vomiting, renal tubular abnormalities, ototoxicity, and hypersensitivity reactions.

Topotecan and CPT-11 are the generic names assigned to Hycamptin® and Camptosar®. These compounds are derivatives of camptothecin. The chemical name for topotecan hydrochloride is (S)-10-[(dimethylamino)methyl]-4-ethyl-4,9-dihydroxy-1H-pyrano[3′,4′:6,7]indolizino[1,2-b]quinoline-3,14-(4H,12H )-dione monohydrochloride. The chemical name for CPT-11 is (4S)-4,11-diethyl-4-hydroxy-9-[(4-piperidinopiperidino)carbonyloxy]-1H-pyrano[3′,4′:6,7]indolizino[1,2-b]quinoline-3,14(4H,12H) dione hydrochloride. Both are commercially available as preparations for IV injection. Topotecan is disclosed in U.S. Pat. No. 5,004,758, which is incorporated herein by reference for its teaching of how to synthesize, formulate, and use topotecan for treating susceptible neoplasms. Similarly, CPT-11 is disclosed in U.S. Pat. No. 4,604,463, which is incorporated herein by reference for its teaching of how to synthesize, formulate, and use CPT-11 for treating susceptible neoplasms. Topotecan and CPT-11 interact with DNA topoisomerase I, resulting in single stranded, and ultimately double stranded breaks in DNA. Topotecan and CPT-11 are used in the treatment of cell lung cancer and ovarian, colorectal, and esophageal cancers. Use of camptothecin analogs is generally accompanied by several side effects including myelosuppression, nausea and vomiting, and hypersensitivity reactions.

Etoposide or VP-16 are the generic names for epipodophyllotoxin. The chemical name for etoposide is 4′-demethylepipodophyllotoxin 9-[4,6-0-(R)-ethylidene-(beta)-D-glucopyranoside]. Etoposide is commercially available as capsules for oral administration or as a solution for IV injection. Etoposide is disclosed in U.S. Pat. No. 3,524,844, which is incorporated herein by reference for its teaching of how to synthesize, formulate, and use etoposide for treating susceptible neoplasms. Etoposide interacts with DNA topoisomerase II resulting in single stranded, and ultimately double stranded breaks in DNA. Etoposide is used in the treatment of small and cell lung cancers, germ cell cancers and lymphomas. Use of etoposide is generally accompanied by several side effects including myelosuppression, nausea and vomiting, hypersensitivity reactions, and mucocutaneous effects.

Doxorubicin is the generic name for Adriamycin®. The chemical name for doxorubicin is 5,12-Naphthacenedione, 10-[(3-amino-2,3,6-trideoxy-(alpha)-L-lyxohexopyranosyl)oxy]-7,8,9,10-tetrahydro-6,8,11-trihydroxy-8-(hydroxylacetyl)-1-methoxy-, hydrochloride (8S-cis). Doxorubicin is commercially available for IV injection. Doxorubicin is disclosed in U.S. Pat. No. 3,590,028, which is incorporated herein by reference for its teaching of how to synthesize, formulate, and use doxorubicin for treating susceptible neoplasms. Doxorubicin binds to nucleic acids, presumably by specific intercalation of the planar anthracycline nucleus with the DNA double helix, resulting in abnormal cellular replication. Doxorubicin is used in the treatment of breast, bladder, liver, lung, prostate, stomach and thyroid cancers; bone and soft tissue sarcomas; lymphomas and leukemias; and tumors of childhood. Use of doxorubicin is generally accompanied by several side effects including myelosuppression, nausea and vomiting, mucocutaneous, and cardiac effects.

Capecitabine is the generic name for Xeloda®. The chemical name for capecitabine is 5′-deoxy-5-fluoro-N-[(pentyloxy)carbonyl]-cytidine. Capecitabine is commercially available as tablets for oral administration. Capecitabine is disclosed in U.S. Pat. Nos. 4,966,891 and 5,472,949, which are incorporated herein by reference for their teaching of how to synthesize, formulate, and use capecitabine for treating susceptible neoplasms. This drug is enzymatically converted to 5-fluorouracil (5-FU) in vivo. Both normal and tumor cells metabolize 5-FU to 5-fluoro-2′-deoxyuridine monophosphate (FdUMP) and 5-fluorouridine triphosphate (FUTP). These metabolites cause cell injury by two different mechanisms. First, FdUMP and the folate cofactor, N5,10-methylenetetrahydrofolate, bind to thymidylate synthase (TS) to form a covalently bound ternary complex. This binding inhibits the formation of thymidylate from 2′-deoxyuridylate. Thymidylate is the necessary precursor of thymidine triphosphate, which is essential for the synthesis of DNA, so that a deficiency of this compound can inhibit cell division. Second, nuclear transcriptional enzymes can mistakenly incorporate FUTP in place of uridine triphosphate (UTP) during the synthesis of RNA. This metabolic error can interfere with RNA processing and protein synthesis. Capecitabine is used in the treatment of breast and colorectal cancers. Use of capecitabine is generally accompanied by several side effects including diarrhea, nausea, vomiting, myelosuppression, stomatitis, and hand-and-foot syndrome.

Radiation therapy is, in many cases, the therapy of choice for the treatment of cancers, including esophageal, mammary, head and neck, brain, prostate and certain leukemias. However, it is well known that incomplete killing of neoplastic cells can result in the recurrence of cancer even after rigorous radiation treatment regimens are completed. Indeed, there are suggestions that some cell populations are stimulated to proliferate as a result of exposure to radiation, thus completely defeating the purpose of the treatment. Clearly, the need for more efficient methods to kill neoplastic cells persists, and a method to eliminate the occurrence of cellular proliferation in response to radiation therapy would be highly beneficial.

In addition, severe side effects are often associated with radiation therapy, including fibrosis, mucocitis, leukopenia and nausea. The development of radiation therapy methods which utilize fewer exposures to radiation, or lower doses per exposure, or both, and yet which still achieve the same or enhanced levels of anti-neoplastic activity, would be highly advantageous.

The molecular mechanism(s) by which tumor cells are killed, survive or are stimulated to proliferate after exposure to ionizing radiation are not fully understood. Several reports have demonstrated that radiation activates multiple signaling pathways within cells in vitro which can lead to either increased cell death or increased proliferation depending upon the dose and culture conditions. [Verheij et al. (1996) Nature, 380, 75-79; Rosette and Karin (1996) Science 274, 1194-1197; Chmura et al. (1997) Cancer Res. 57, 1270-1275; Santana et al. (1996) Cell 86, 189-199; Kyriakis and Avruch (1996) Bioessays 18, 567-577; Xia et al. (1995) Science 270, 1326-1331; Kasid et al. (1996) Nature 382, 813-816]. It has been shown that radiation-mediated activation of acidic sphingomyelinase generates ceramide and subsequently activates the Stress Activated Protein (SAP) kinase pathway (sometimes referred to in the literature as the c-Jun NH.sub.2-terminal kinase (JNK) pathway). This pathway has been proposed to play a major role in the initiation of apoptosis (cell death) by radiation (Verheij et al.; Rosette et al.; Chmura et al.; Santana et al.; Kyriakis and Avruch; Xia et al.).

With respect to the cellular response to ionizing radiation, another cellular target has been proposed to be involved. The epidermal growth factor (EGF) receptor has been shown to be activated in a dose dependent fashion in response to radiation [Schmidt-Ullrich et al. (1996) Radiation Research, 145, 81-85; Schmidt-Ullrich et al. (1997) Oncogene 15, 1191-1197].

Among the newer chemotherapeutic agents being developed are target specific chemical entities. Since EGF has been associated with certain tumor types and with cell proliferation, a number of agents are been developed which inhibit the EGF receptor tyrosine kinases. The EGF receptor tyrosine kinase family includes the erbB receptor kinases erbB1, erbB2, erbB3, and erbB4. Most of these erbB tyrosine kinase inhibitors are reversible inhibitors. They bind to the receptor and are released. In addition most of these tyrosine kinase inhibitors are specific for only one of the kinases in the erbB receptor tyrosine kinase family. However, U.S. Pat. Nos. 6,344,455 and 6,344,459 describe irreversible inhibitors of erbB receptor tyrosine kinases erbB1, erbB2, erbB3, and erbB4, i.e., PAN erbB receptor tyrosine kinase inhibitors. The preferred PAN erb B tyrosine kinase inhibitor is N-[4-(3-chloro-4-fluoro-phenylamino)-7-(3-morpholin4-yl-propoxy)-quinazolin-6-yl]-acrylamide. It is also known as CI-1033. It is described in WO 00/31048, which is incorporated herein by reference for its teaching of how to make N-[4-(3-chloro-4-fluoro-phenylamino)-7-(3-morpholin-4-yl-propoxy)-quinazolin-6-yl]-acrylamide, how to formulate it into dosage forms, and how to use it for treating cancers and other cell proliferative disorders.

### SUMMARY OF THE INVENTION

This invention relates to a synergistic combination of antineoplastic agents, and to a method for treating tumors comprising administering to a patient an erb B inhibitor in a therapeutic regimen with at least one other chemotherapeutic agent or with radiation therapy. Preferably, the erb B inhibitor is an irreversible inhibitor of at least one receptor of the erb B family of tyrosine kinases. More preferably the erb B inhibitor is a PAN erb B tyrosine kinase inhibitor. Most preferably, the erb B inhibitor is an irreversible PAN erb B tyrosine kinase inhibitor. The preferred irreversible PAN erb B tyrosine kinase inhibitor is N-[4-(3-chloro-4-fluoro-phenylamino)-7-(3-morpholin-4-yl-propoxy)-quinazolin-6-yl]-acrylamide (CI-1033). The invention more particularly provides a therapeutic regimen comprising, as one component, CI-1033, and a second component selected from the group consisting of gemcitabine, paclitaxel, docetaxel, cisplatin, carboplatin, etoposide, adriamycin, topotecan, CPT-11, capecitabine, and ionizing radiation. The invention also provides a therapeutic regimen comprising at least one erb B kinase inhibitor and at least one other chemotherapeutic agent.

### DESCRIPTION OF FIGURES

FIG. 1 shows the synergy of CI-1033 and Taxotere® in human H125 non-small lung cell carcinoma xenografts.

FIG. 2 shows the synergy of CI-1033 and radiation in a murine Rif-1 sarcoma.

FIG. 3 demonstrates the schedule dependence of Taxol when combined with CI-1033 on MDA-MB-468 Breast cancer cells.

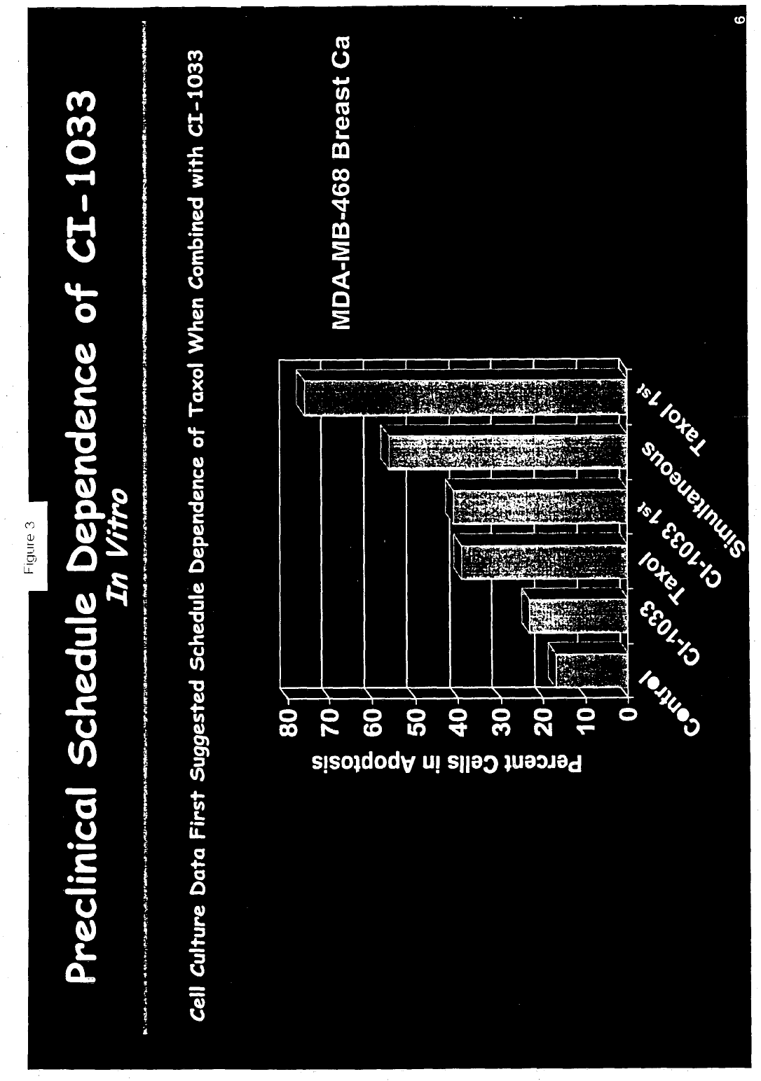

FIG. 4 demonstrates the in vivo schedule dependence observed when CI-1033 is combined with Cisplatin.

### DETAILED DESCRIPTION OF THE INVENTION

It is an object of this invention to provide a method to delay growth or kill hyperproliferating cells, comprised of exposing the hyperproliferating cells to an inhibitor of at least one erb B kinase in combination therapy with another conventional antineoplastic agent. Preferably, the erb B kinase inhibitor is an irreversible erb B inhibitor. More preferably, the erb B kinase inhibitor inhibits more than one erb B kinase. It is a further object of this invention to provide a method to treat hyperproliferative cell disorders such as, but not limited to, cancer in mammals. That method will encompass administering, a lethal agent (e.g. ionizing radiation, chemotherapeutic agents, heat, ultraviolet light, high intensity red light as used in photo-dynamic therapy, etc.) in combination therapy with an inhibitor, preferably, an irreversible inhibitor of the erb B tyrosine kinases. The administration of such an erbB tyrosine kinase inhibitor potentiates the ability of radiation or chemotherapy, or both, or of other lethal agents, to cause apoptosis of cancer cells, thus stabilizing disease progression and decreasing cancer recurrences.

The invention contemplates the use of any pan erb B tyrosine kinase inhibitor, and preferably an irreversible PAN erb B tyrosine kinase inhibitor. The preferred irreversible PAN erb B tyrosine kinase inhibitor is N-[4-(3-chloro-4-fluoro-phenylamino)-7-(3-morpholin-4-yl-propoxy)-quinazolin-6-yl]-acrylamide, an irreversible erbB inhibitor. It is also known as CI-1033. CI-1033 is described in WO 00/31048, which is incorporated herein by reference for its teaching of how to make N-[4-(3-chloro-4-fluoro-phenylamino)-7-(3-morpholin-4-yl-propoxy)-quinazolin-6-yl]-acrylamide, how to formulate it into dosage forms, and how to use it for treating cancers such as colon, breast, ovarian, pancreatic, prostate, lung cancer, other cancers such as adenocarcinomas and sarcomas.

Administration of the PAN erb B tyrosine kinase inhibitor may be, for example, prior to, after, or concurrent with the radiation or chemotherapy treatment. One skilled in the art will recognize that the amount of PAN erb B tyrosine kinase inhibitor to be administered will be that amount sufficient to enhance the anti-neoplastic effect of the radiation and/or chemotherapy. Such an amount may vary inter alia depending on the gender, age, weight and condition of the patient, and must be determined on a case by case basis. The amount may vary according to the size and type of neoplasia, as well as the particular radiation or chemotherapy protocol that is followed. Generally, a suitable dose is one that results in a concentration of the inhibitor at the site of the tumor in the range of 0.5 nM to 200 μM, and more usually from 20 nM to 80 nM. It is expected that serum concentrations from 40 nM to 150 nM should be sufficient in most cases. Administration may be oral, parenteral or topical, and is likely to be oral or intravenous. The inhibitor may be administered in any of several forms, including tablets, pills, powders, lozenges, sachets, cachets, elixirs, suspensions, emulsions,.solutions, syrups, aerosol (as a solid or in a liquid medium), soft or hard gelatin capsules, suppositories, sterile injectable solutions and sterile packaged powders for either oral or topical application.

The compositions useful in practicing this invention comprise the above active ingredients, or suitable salts thereof, together with common excipients, diluents, and carriers.

A preferred treatment comprises CI-1033, used in conjunction with one or more of gemcitabine, paclitaxel, docetaxel, cisplatin, carboplatin, etoposide, adriamycin, topotecan, CPT-11, or capecitabine. Another preferred combination is use of CI-1033 in a protocol for treatment of cancers with ionizing radiation. In another preferred embodiment is a method of treating cancers comprising administering CI-1033 in a protocol with ionizing radiation and another antineoplastic agent selected from the group consisting of gemcitabine, paclitaxel, docetaxel, cisplatin, carboplatin, etoposide, adriamycin, topotecan, CPT-11, or capecitabine.

The present invention provides a unique combination of antineoplastic agents that exhibits a dramatic synergistic effect. The combination utilizes an irreversible PAN erb B tyrosine kinase inhibitor, in conjunction with the administration of cytotoxic agents such as gemcitabine, paclitaxel, docetaxel, or a protocol for use with ionizing radiation therapy. These combinations are especially effective in treating patients with solid tumors, especially cell lung cancer and other advanced solid tumors.

An object of this invention is to provide a method for treating hyperproliferative cell disorders with a combination comprising CI-1033 together with at least one of either gemcitabine, paclitaxel, taxotere, cisplatin, carboplatin, etoposide, adriamycin, topotecan, CPT-11, capecitabine or ionizing radiation. The term hyperproliferative cell disorder includes such disorders as psoriasis, cancer, and restenosis. A further object is to provide a composition comprising synergistic amounts of CI-1033 and gemcitabine, synergistic amounts of CI-1033 and paclitaxel, synergistic amounts of CI-1033 and taxotere, synergistic amounts of CI-1033 and cisplatin, synergistic amounts of CI-1033 and carboplatin, synergistic amounts of CI-1033 and etoposide, synergistic amounts of CI-1033 and adriamycin, synergistic amounts of CI-1033 and topotecan, synergistic amounts of CI-1033 and CPT-11, synergistic amounts of CI-1033 and capecitabine, and a synergistic amount of CI-1033 to be used with ionizing radiation.

In a further embodiment of the invention, we provide a method for treating cancer comprising administering to an animal in need of treatment an effective amount of a combination of CI-1033 and at least one therapy selected from the group consisting of ionizing radiation, gemcitabine, paclitaxel, docetaxel, cisplatin, carboplatin, etoposide, adriamycin, topotecan, CPT-11, and capecitabine.

A preferred method embraces treatment of solid tumors with the combinations comprising CI-1033 and conventional antineoplastic therapeutic modalities.

A further preferred method employs an antitumor amount of CI-1033 and an effective amount of at least one of gemcitabine, paclitaxel, docetaxel, cisplatin, cisplatin, carboplatin, etoposide, adriamycin, topotecan, CPT-11, or capecitabine, or ionizing radiation to treat susceptible cancers, including cell lung cancer (NSCLC), breast cancer, ovarian cancer, head and neck cancer, myelomas, prostate cancer, colon cancer, pancreatic cancer and other solid tumors. In another embodiment, CI-1033 may be used in the present invention in combination with two or more other antineoplastic therapeutic modalities.

Another embodiment of the invention is a kit comprising in one compartment a dosage of CI-1033, and in another compartment a dosage of an agent selected from the group consisting of gemcitabine, paclitaxel, docetaxel, cisplatin, carboplatin, etoposide, doxorubicin, topotecan, CPT-11, capecitabine, or a pharmaceutically acceptable salt thereof. In another embodiment, the kit comprises a dosage of CI-1033 and dosages of at least two compounds selected from the group consisting of gemcitabine, paclitaxel, docetaxel, cisplatin, carboplatin, etoposide, adriamycin, topotecan, CPT-11, or capecitabine. Included in the kit are also instructions for use of the combinations of the present invention, including directions for dosing, dosage schedules and preparation and administration of the agents used in the combination.

The compounds utilized in the method of this invention may be administered in doses commonly employed clinically. Depending on the stage of the disease, the tumor type and the general condition of the mammal in need of such treatment, lower doses of each of the antineoplastic modalities than are conventionally administered may be used to achieve similar efficacy against the tumor then are conventionally used as a single agent while also diminishing the side effects. Such doses can be calculated in the normal fashion, for example on body surface area. CI-1033 is administered, for example, at doses from about 10.0 mg to about 200 mg for continuous dosing, preferably from about 50.0 mg to about 200.0 mg. Ideally, CI-1033 will be administered at a dose that will produce plasma levels of about 5 to about 100 μg/mL. CI-1033 typically is administered orally, for example, as capsules having active ingredient in the amounts of 5, 25, 50, 75, 100, and 200 mg per capsule. CI-1033 is administered daily at about the same dose levels throughout a treatment period, typically for 15 to 30 days. Alternatively, the daily dosage of CI-1033 may be administered in divided doses during a 24 hr period. Multiple treatment periods can be practiced, as dictated by the attending medical practitioner and the particular patient and condition being treated. Intravenous administration of CI-1033 is also contemplated when warranted by the medical condition of the patient or to comport with other concurrent medical treatments.

Gemcitabine is administered at doses comparable to those routinely utilized clinically. For example, the initial dose of gemcitabine, typically as the hydrochloride salt, is about 1000 mg/m² of body surface area. Gemcitabine is routinely formulated as a sterile solution and is administered by intravenous infusion, generally over about a 30-minute period, with about 2 to 4 weekly doses, with courses repeated about every 28 to 30 days. The dose of 1000 mg/m² can be given for up to about 7 weeks, according to this treatment regimen, or until undesirable side effects are observed. Other salt forms can be utilized if desired, for example, the hydrobromide, monophosphate, sulfate, malonate, citrate, and succinate are readily prepared.

Capecitabine, for monotherapy, generally is administered orally at a dose of about 2500 mg/m² daily for 2 weeks, followed by a 1-week rest period. The product is supplied commercially in 150 mg and 500 mg tablets. The tablets are administered at the rate of about 1 to about 4 times a day during the treatment period.

Paclitaxel, or docetaxel generally are formulated as sterile solutions for injection, and routinely administered at doses of about 60 to 175 mg/m², given intravenously, on a daily basis or intermittent basis. Paclitaxel may also be administered at a dose of 135-175 mg/m² intravenously over a 3-hour infusion or for docetaxel IV at a dosage of 60-100 mg/m² for 1 hour. Alternatively, ionizing radiation may be administered as a single dose of from about 2.5 to 56 Gy. Ionizing radiation may be administered as a single dose repeated at long time intervals or divided into more frequent smaller doses. This cycle can be repeated for about every 4 to 8 weeks.

Cisplatin is formulated as a sterile solution;for injection, and is routinely administered intravenously at a dose of approximately 20 mg/m² daily for 5 days or at 75-100 mg/² every 4 weeks.

Carboplatin is formulated as a sterile powder that is reconstituted prior to IV injection, and is routinely administered intravenously at a dose of approximately 360 mg/m² every 4 weeks.

Topotecan is formulated as a sterile powder that is reconstituted prior to IV injection, and is routinely administered intravenously at a dose of approximately 1.5 mg/m² every 3 weeks

CPT-11 is formulated as a sterile liquid that is diluted prior to IV injection, and is routinely administered intravenously at a dose of 50-150 mg/m² weekly for 4 weeks, followed by a 2-week rest period.

Etoposide is formulated as a sterile liquid which is diluted prior to IV injection, and is routinely administered intravenously on several different treatment schedules including 120 mg/m² IV on days 1-3 repeated every 21 days, 50-100 mg/m² IV on days 1-5 every 2-4 weeks, 125-140 mg/m² on days 1, 3, 5 every 3-5 weeks. Dosing may also consist of etoposide tablets/capsules at 50 Mg/m² for 21-days every 4-5 weeks.

Doxorubicin is formulated as a sterile powder that must be reconstituted and diluted prior to IV administration. Doxorubicin is administered intravenously at 60-75 mg/m² every 3 weeks, 15-20 mg/m² weekly, or 30 mg/m² on days 1 and 8 every 4 weeks.

Doses and administration schedules of these agents may vary in combination chemotherapy protocols. In addition, salts, other than those specifically listed may be used in combination therapeutic protocols. Those skilled in the art will recognize that these combinations are exemplary only, and that related compounds or derivatives of these antineoplastic agents may be used in combination with the reversible or irreversible erb B tyrosine kinase inhibitor.

The combinations provided by this invention have been evaluated in vivo against several different in vivo tumor models. The combination experiments of CI-1033 with radiation were performed in two different in vivo tumor models. The combination chemotherapy experiments were performed using five different in vivo tumor models and seven different chemotherapeutic agents.

While the results exemplify the use of CI-1033, an irreversible PAN erb B tyrosine kinase inhibitor, similar results may be obtained with other agents that inhibit these kinases.

CI-1033 was administered clinically in doses ranging from 50 mg to 750 mg/day when the duration of treatment was 14 consecutive days. The treatment may be prolonged, with or without an off drug rest period. Lower doses of CI-1033 were used for 8 weeks of continuous daily therapy, followed by a 2-week ‘drug holiday’. With CI-1033 alone, there was no evidence of cumulative toxicity following repeated courses and prolonged exposures to CI-1033. In preliminary studies with CI-1033 alone, responses included one partial response in a heavily pretreated patient with NSCLC and a minor response in one patient each with renal cell cancer and NSCLC.

The following detailed examples further establish the synergy between CI-1033 and either gemcitabine, paclitaxel, docetaxel, cisplatin, carboplatin, etoposide, doxorubicin, topotecan, CPT-11, capecitabine or ionizing radiation. These Examples are exemplary only and are not intended to limit the scope of the invention.

### EXAMPLE 1

### Anticancer Effectiveness of Combination Chemotherapy with CI-1033 and Gemcitabine Against Orthotopically Implanted L3.6pl Human Pancreatic Carcinoma in Nude Mice

The synergistic combinations provided by this invention have been evaluated in standard chemotherapy studies using female immunodeficient nude mice. The combination of CI-1033 with gemcitabine was evaluated against an orthotopically implanted human pancreatic xenograft.

L3.6pl human pancreatic cancer cells were established from COLO 357 fast growing cells by injecting them into the pancreas of nude mice, with subsequent harvesting of hepatic metastases and re-implantation into the pancreas for three cycles. The resulting L3.6pl cells produce a significantly higher incidence of hepatic and lymph node metastases than the parental cells. Cells were maintained on plastic in Dulbecco's Modified Eagle's medium (DMEM) supplemented with 5% fetal bovine serum (FBS), sodium pyruvate, non-essential amino acids, L-glutamine, and 2-fold vitamin solution GIBCO, Grand Island, NY), incubated in 5% CO₂-95% air at 37 degrees C. Cultures were maintained for no more than 8-weeks after recovery from frozen stocks.

Animals and Orthotopic Implantation of Tumor Cells.

Male athymic BALB/c nude mice were from the National Cancer Institute-Frederick Cancer Research and Development Center (Frederick, Md.). Mice were housed and maintained in laminar flow cabinets under pathogen free conditions approved by the American Association for the Accreditation of Laboratory Animal Care, and their use in these experiments approved by the Institutional Animal Care and Use Committee.

To produce tumors, cells were harvested from subconfluent cultures by treatment with 0.25% trypsin and 0.2% EDTA. Trypsinization was stopped with medium containing 10% FBS, and the cells washed once with serum free medium and resuspended in Hank's Balanced Salt Solution (HBSS) at a concentration of 2×10⁷ cells/mL. Only single cell suspensions with greater than 90% viability were used for injection. Mice (8-10 weeks of age) were anesthetized with methyoxyflurane, the pancreas exposed and 1×10⁶ cells in 0.05 mL injected into the body of the pancreas. Incisions were closed with wound clips. Mice were sacrificed after 5-6 weeks of tumor growth. The size and weight of primary tumors and the incidence of lymph node and hepatic metastases were determined at the time of sacrifice.

Treatment of Established Human Pancreatic Carcinoma Xenografts with CI-1033 and Gemcitabine.

Mice were implanted with 1×10⁶ L3.6pl human pancreatic carcinoma cells intrapancreatically on day 0. Therapy was initiated on day 7 post tumor cell implant. The duration of therapy was four weeks. Pancreas weight, tumor weight and incidence of metastasis were recorded at the time of terminal sacrifice. Gemcitabine (125 mg/kg) was administered intraperitoneally in 0.5 mL saline twice weekly for 4-weeks. CI-1033 was administered orally, once daily, 5-days per week for 4-weeks at 30 mg/kg (high dose) and 10 mg/kg (low dose). The study consisted of six treatment groups with a minimum of 10 mice per treatment group. Groups were control, gemcitabine alone, CI-1033 at 30 mg/kg alone, CI-1033 at 10 mg/kg alone, gemcitabine plus CI-1033 at 30 mg/kg, and gemcitabine plus CI-1033 at 10 mg/kg.

Control animals lost 17% of their initial body weight by the end of the four week therapy period. At terminal sacrifice, the control animals had lost 24% of their initial weight. Weight loss in this group is attributed to pancreatic carcinoma progression. Tumor bearing animals treated with gemcitabine alone at 125 mg/kg twice weekly had a slight weight gain over the therapy period, but had an overall 4% loss of initial body weight at terminal sacrifice. Mice dosed with 10 and 30 mg/kg CI-1033 lost 6 and 9% of their initial body weight during therapy, respectively, but gained weight in the period between the end of therapy and terminal sacrifice. The CI-1033 (10 mg/kg) plus gemcitabine treated group lost 10% of initial body weight during therapy, but recovered the lost body weight after the end of therapy. At the end of the second week of dosing the CI-1033 (30 mg/kg) plus gemcitabine treatment group had a body weight loss of 16%. Because of the large weight loss this combination dosage group was given a drug free holiday during therapy week three, with dosing reinitiated in week four of the study.

The trend in antitumor effectiveness was the same whether examining total pancreas mass or tumor volume. The combination groups showed improved efficacy compared to the groups treated with only gemcitabine or CI-1033 suggesting that the combination improved upon antitumor effectiveness over that obtained with single agent therapy. The rank order of therapeutic effectiveness was CI-1033 (30 mg/kg) plus gemcitabine>CI-1033 (10 mg/kg) plus gemcitabine>CI-1033 (30 mg/kg)=gemcitabine>CI-1033 (10 mg/kg). The rank order was the same when calculating percent T/C values based on tumor volume. None of these treatment regimens produced a reduction in lymph node metastases. However, CI-1033 appeared to reduce the number of hepatic metastases over that obtained with gemcitabine. The combination of CI-1033 and gemcitabine produced an antitumor effect that was superior to that produced by either of the single agents alone.

### EXAMPLE 2

### Anticancer Effectiveness of Combination Chemotherapy with CI-1033 and Paclitaxel Against Orthotopically Implanted 253J B-V Human Bladder Carcinoma in Nude Mice

The synergistic combinations provided by this invention have been evaluated in standard chemotherapy studies using female immunodeficient nude mice. The combination of CI-1033 and paclitaxel was evaluated against an orthotopically implanted human transitional cell (bladder) xenograft. The highly metastatic human transitional cell carcinoma 253J B-V was maintained as a monolayer culture in modified Eagle's minimal essential medium supplemented with 10% fetal bovine serum, vitamins, sodium pyruvate, L-glutamine, and non-essential amino acids as described previously.

Animals and Orthotopic Implantation of Tumor Cells.

Male athymic BALB/c nude mice were from the National Cancer Institute-Frederick Cancer Research and Development Center (Frederick, Md.). Mice were housed and maintained in laminar flow cabinets under pathogen free conditions approved by the American Association for the Accreditation of Laboratory Animal Care, and their use in these experiments approved by the Institutional Animal Care and Use Committee.

To produce tumors, cells were harvested from subconfluent cultures by treatment with 0.25% trypsin and 0.2% EDTA. Trypsinization was stopped with medium containing 10% FBS, and the cells washed once with serum free medium and resuspended in Hank's Balanced Salt Solution (HBSS) at a concentration of 2×10⁷ cells /mL. Only single cell suspensions with greater than 90% viability were used for injection. Mice (8-10 weeks of age) were anesthetized with methyoxyflurane to effect, a lower midline incision made, and the bladder exposed. Viable tumor cells (1×10⁶ cells in 0.05 mL) were injected into the wall of the bladder. Incisions were closed with wound clips. Mice were sacrificed after 6 weeks of tumor growth. The size and weight of primary bladder tumors were recorded at the time of sacrifice.

Treatment of Established Human Bladder Carcinoma Xenografts with CI-1033 and Paclitaxel.

Mice were implanted with 1×10⁶ 253J B-V human bladder carcinoma cells into the bladder wall on day 0. Therapy was initiated on Day 14 post cell implant. The duration of therapy was 4 weeks. Bladder tumor weights were recorded at terminal sacrifice. Paclitaxel (8 mg/kg) was administered intraperitoneally in 0.5 mL on days 14, 20, and 27 post tumor cell implant. CI-1033 was administered orally, either once daily, 5-days per week for 4-weeks at 30 mg/kg (high dose) and 10 mg/kg (low dose) or twice weekly for 4-weeks at 30 mg/kg. The first study consisted of six treatment groups with a minimum of 6 mice per treatment group. Groups were control, paclitaxel alone, CI-1033 at 30 mg/kg alone, CI-1033 at 10 mg/kg alone, paclitaxel plus CI-1033 at 30 mg/kg, and paclitaxel plus CI-1033 at 10 mg/kg. In the first study CI-1033 was administered orally 5-days per week for 4-weeks). The second study was also composed of 6-groups with a minimum of 8 mice per group. Groups in the second study were control, paclitaxel alone, CI-1033 (30 mg/kg) alone dosed 5-days per week, CI-1033 (30 mg/kg) dosed twice weekly, paclitaxel plus CI-1033 (30 mg/kg) 5-days per week, and paclitaxel plus CI-1033 (30 mg/kg) twice weekly.

Animals in the control groups of both of these studies lost between 1% to 7% of their initial body weight. Single agent therapy with either CI-1033 or paclitaxel induced no more than a 5% reduction in body weight, suggesting that single agent therapy lacked significant toxicity, based on weight loss. Weight loss in combination therapy groups was in the range of 2% to 6%, except for the daily CI-1033 (30 mg/kg) plus paclitaxel treatment group which lost 10% of initial body weight during the first two weeks of therapy. This treatment group recovered a great deal of the weight lost during the last two weeks of therapy for a weight loss from initial dose to sacrifice of 3%. The weight loss in the combination groups suggest that combination therapy with CI-1033 plus paclitaxel did not potentiate toxicity over that observed with the single agents alone on the doses and schedules used in these studies.

The control group and the group dosed IP with 8 mg/kg paclitaxel alone on days 14, 20 and 27 did not significantly reduce the bladder tumor mass. CI-1033 was administered orally on days 14-18, 21-25, 28-32 and 35-39 or on Days 14, 17, 21, 24, 28, 31, 35, and 38 at dosages of 10 or 30 mg/kg. In groups treated with 10 or 30 mg/kg of CI-1033 alone the tumor mass (measured at terminal sacrifice) was reduced to 42% and 25%, respectively of that of the control group. When CI-1033 (at 10 or 30 mg/kg) was administered orally in combination with paclitaxel (8 mg/kg, IP), the tumor masses were further reduced to 22% and 14% of the control group tumor mass.

CI-1033 as a single agent either on a daily or intermittent treatment schedule was at least as effective as paclitaxel in both studies. Intermittent dosing of CI-1033 at 30 mg/kg was as effective as daily dosing at the same dose level.

The combination of paclitaxel and 10 mg/kg or 30 mg/kg CI-1033 daily reduced tumor mass to a greater extent than either single agent alone, resulting in percent T/C values of 25% and 14%, respectively. Results of the second study suggested also that on both treatment schedules CI-1033 plus paclitaxel produced improved antitumor effectiveness over single agent therapy.

Overall, these data indicate that the combination of paclitaxel and CI-1033 was superior in reducing tumor mass than either agent administered as a single agent in these studies against the 253J B-V human bladder carcinoma.

CI-1033/Docetaxel Combination with Sequential Dosing

A number of preclinical studies indicate that a greater therapeutic effect can be obtained by combining inhibitors of the erbB family receptor tyrosine kinases with paclitaxel than by using either agent alone. This result has previously been reported for Iressa™ in several human tumor xenografts including the A431 human epidermoid, the LX-1 lung, the A549 lung and the GEO colon carcinomas. [Sirotnak, et al. Clin. Cancer Res. 6:4885-92, 2000; Ciardiello, et al. Clin. Cancer Res. 7:1459-65, 2001; Ciardiello, et al.Clin. Cancer Res.6:2053-63, 2000.] Monoclonal antibodies directed against individual receptors of the erbB family have also been shown to be effective in combination with this drug. Herceptin™, which specifically neutralizes erbB-2, enhanced the activity of paclitaxel in vivo against the BT-474 human breast carcinoma [Baselga J, et al., Cancer Res. 58:2825-31, 1998.] as well as in a variety of human tumor cell lines in vitro [Pegram, et al., Oncogene 18:2241-51, 1999.] and C-225, which is directed against the EGF receptor has been shown to enhance the antitumor effects of paclitaxel in the 253JB-V human bladder grown orthotopically in athymic nude mice. [Inoue, Clin. Cancer Res. 6:4874-84, 2000.]

However, none of the inhibitors of the erbB family tyrosine kinases described above are irreversible and are pan erbB tyrosine kinase inhibitors. CI-1033 has been shown above to enhance the therapeutic effects when used in combination with paclitaxel. Experiments described below demonstrate that a specific dose sequence enhances the activity of the two drugs in combination. In vitro experiments in which MDA-MB-453 human breast carcinoma cells were exposed to paclitaxel and CI-1033 either alone or in combination have shown an enhancement of paclitaxel-induced apoptosis where maximal effects were dependent on exposure to paclitaxel first. In these experiments, a 3-day exposure to paclitaxel alone induced 23% of the cells to undergo apoptosis, whereas CI-1033 alone did not affect the apoptotic fraction. Combined simultaneous exposure to paclitaxel and CI-1033 resulted in only a marginal increase in cell death to 27%. However, if the paclitaxel was added first, followed by CI-1033 at 24 hours later, the apoptotic fraction was doubled to 47%. In contrast, if the cells were exposed to CI-1033 24 hours prior to paclitaxel, apoptosis was markedly suppressed to only 6%. In vivo efficacy tests in the A431 human tumor xenograft with CI-1033 in combination with paclitaxel have shown that initial treatment with paclitaxel first followed one day later with CI-1033 was a highly efficacious schedule in which the combination produced a greater therapeutic effect than either drug alone. Furthermore, the combination was well tolerated and there appeared to be no overlapping toxicities. These results are consistent with the enhanced activity of paclitaxel produced in combination with the EGF receptor antibody, C225, when the antibody was given 2 days after the chemotherapy. [Inoue, Clin. Cancer Res. 6:4874-84, 2000.]

The antitumor activity observed with combinations of the EGF receptor antibody C225 and topotecan showed clear sequence-dependence where the greatest effect was obtained when topotecan was given first followed one day later with the antibody. Activity was less when the two drugs were given simultaneously and markedly suppressed when C225 was given first. [Ciardiello, et al. Clin. Cancer Res. 5:909-16, 1999.]

Studies with CI-1033 have also shown marked sequence dependent effects with 2 additional drugs. Enhanced cell kill was observed in vitro by exposing cells initially to gemcitabine followed by CI-1033 [Nelson, et al., J. Biol. Chem. 276:14842-14847, 2001] similar to the paclitaxel studies described above. In vivo tests in the A431 human epidermoid carcinoma with CI-1033 have also shown striking sequence dependence with cis-platin, in which dosing CI-1033 subsequent to cis-platin provided a greater therapeutic effect but pre-dosing CI-1033 inhibited activity.

Collectively, these data imply that CI-1033 should not be given prior to the docetaxel and although simultaneous administration may provide benefit, the greatest antitumor effect can potentially be obtained by sequential dosing with prior treatment of docetaxel followed by CI-1033.

### EXAMPLE 3

### Design of Growth Delay (T-C) Trials

The synergistic combinations provided by this invention have been evaluated in standard chemotherapy studies using female conventional immunodeficient nude mice weighing 18 to 20 grams. On Day 0 of the test, each mouse was surgically implanted (subcutaneously) with a fragment of tumor weighing approximately 30 mg. The mice were weighed weekly, and tumor size (width and length in mm) were measured three times each week with standard calipers. Tumor mass for each animal was calculated according to the formula:

$$mass = \frac{( a \times {b}^{2} )}{2} ,$$

where “a” is width of the tumor in mm, and “b” is the length in mm. Evaluation of anticancer activity was evaluated based on the formula T-C, where “T” and “C” are the median time (in days) required for the treated and control (respectively) tumors to reach a pre-determined size of 750 mg (the “evaluation size”). CI-1033 was dissolved in 50 mM sodium lactate buffer, pH 4.0, and administered orally at various dosages in 0.5 mL volumes. Standard agents were diluted as described in the package inserts and administered at various dosage levels in 0.5 mL injections.

In each experiment, mice bearing established tumors were randomized into one of four treatment groups. One group served as control treatment groups. Group 2 was further divided into four sub-groups, each of which received oral doses of CI-1033 at a specified level of active drug. The CI-1033 was administered according to the schedules indicated below. The third group was further divided into four subgroups, each of which received the designated standard agent by the route and schedules indicated below.

Group 4 was further subdivided into groups receiving combination therapy. Each dose of CI-1033 was evaluated with each dosage level of standard chemotherapeutic.

The data presented from the orthotopic tumor model studies In Examples 1 and 2 establish that the combination of CI-1033 and gemcitabine or paclitaxel is surprisingly active in reducing the rate of growth of tumors in animals. The ability of these agents when used together establish the combination to be superior as an antitumor agent than either of the agents used alone.

### EXAMPLE 4

### Tumor Growth Delay with CI-1033 in Combination with Docetaxel

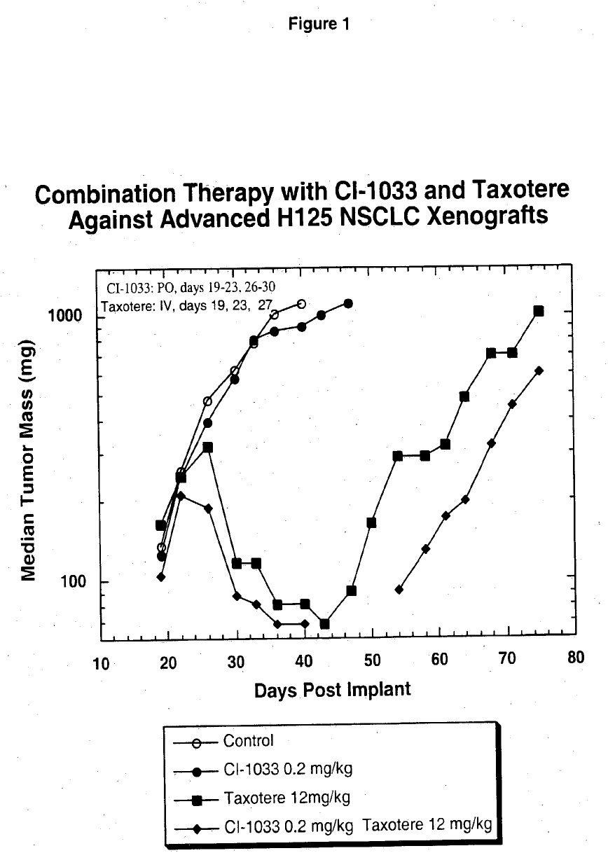

Because the synergistic effects observed with the combination of CI-1033 and paclitaxel were so surprisingly dramatic, a tumor growth delay study with docetaxel and CI-1033 was conducted against a subcutaneously implanted human non-small cell lung cancer xenograft, H125. CI-1033 at 40, 10, 2.5, 0.7 and 0.2 mg/kg was administered PO on days 19-23 and 26-30 to mice having established H125 human cell lung carcinoma xenografts. Docetaxel at doses of 12, 8, and 5 mg/kg was administered IV on days 19, 23, and 27. The optimum tumor growth delays for CI-1033 and docetaxel as single agents were 11.7 and 35.7 days, respectively. Several of the groups given combination chemotherapy demonstrated tumor growth delays in excess of 35 days indicating an enhanced therapeutic benefit for the combination therapy comprising docetaxel and CI-1033. FIG. 1 demonstrates the enhanced tumor growth delay accompanying treatment with CI-1033 and docetaxel. Complete responses were defined as tumors that decreased in mass by 100% during the study. Partial responses were defined as tumors that decreased in mass by at least 50% during the study. The number of partial and complete responses observed in animals receiving both therapeutic agents in this study was higher for those animals receiving combined therapy than for those receiving single agent therapy. However, the number of complete responses observed in animals receiving both therapeutic agents in this study was markedly elevated over those receiving single agents (13.3% vs 4% and 0). The combination of these two agents did not effect toxicity, lethality or weight loss.

### EXAMPLE 5

### Tumor Growth Delay with CI-1033 in Combination with Etoposide

The synergistic combinations provided by this invention have been evaluated in standard chemotherapy studies using female immunodeficient nude mice. The combination of CI-1033 and etoposide was evaluated against a subcutaneously implanted human non-small cell lung cancer xenograft, H125.

In one combination trial with CI-1033 and etoposide, CI-1033 at doses of 200, 124, and 77 mg/kg was administered 24 hours after each of 3 etoposide doses. Etoposide was administered IP at doses of 80, 50, and 31 mg/kg on days 12, 16 and 20. Etoposide was relatively ineffective in delaying the growth of H125 as a single agent at a maximum tolerated dose of 50 mg/kg in this trial while CI-1033 was very effective. The combination of etoposide at 50 mg/kg and CI-1033 at 77 mg/kg produced a superior effect in delaying tumor growth than that observed with either single agent administered alone. All other combination dosage regimens were no better than CI-1033 therapy alone. However, etoposide was well tolerated only at the lowest dose tested.

### EXAMPLE 6

### CI-1033 in Combination with Capecitabine

The synergistic combinations provided by this invention have been evaluated in standard chemotherapy studies using BALB/C female mice. The combination of CI-1033 and capecitabine was evaluated against a subcutaneously implanted murine colon carcinoma, C26.

In one combination trial with CI-1033 and capecitabine, CI-1033 at doses of 40, 20, and 10 mg/kg was administered orally simultaneously with each capecitabine dose. Capecitabine was administered PO at doses of 750 and 500 mg/kg on days 14-16, 21-23, and 28-30. The optimum tumor growth delays for CI-1033 and capecitabine as single agents were 3.6 and 22.5 days, respectively. Several of the groups given combination chemotherapy demonstrated tumor growth delays in excess of 22 days indicating an enhanced therapeutic benefit for the combination therapy comprising capecitabine and CI-1033.

Capecitabine caused 3/6 complete responses and 2/6 partial responses against C26 colon carcinoma as a single agent, while CI-1033 as a single agent was ineffective in this trial. The combination of capecitabine at 750 or 500 mg/kg and CI-1033 produced a greater than additive effect (14/36 (39%)) complete responses and (5/36 (14%)) partial responses. As single agents CI-1033 and capecitabine produced 0 and 8% tumor free survivors, respectively. Combinations of CI-1033 and capecitabine produced 16% tumor free survivors.

Thus, this experiment demonstrates that the combination of capecitabine and CI-1033 administered to mice bearing advanced murine colon 26/clone 10 produced superior anticancer effectiveness compared to either of the single agents alone.

### EXAMPLE 7

### CI-1033 in Combination with Cisplatin

The combination of CI-1033 and cisplatin was evaluated in immunodeficient female nude mice against a subcutaneously implanted human non-small cell lung cancer xenograft, H125.

CI-1033 at 40, 20, 10, 5 and 2.5 mg/kg was administered PO on days 28-37 to mice having advanced OVCAR-5 Human Ovarian Cancer xenografts. Cisplatin at doses of 12, 6, 3, and 1.5 mg/kg was administered IV on days 28, 32, and 34. Neither cisplatin nor CI-1033 when administered alone produced meaningful anticancer effects against advanced OVCAR-5 human ovarian cancer xenografts. In this trial CI-1033 administered following cisplatin therapy provided superior anticancer effects against advanced OVCAR-5 than either single agent, i.e., for instance tumor growth delays (T-C's) greater than 18 days for combinations of CI-1033 at 5 and 10 mg/kg with 12 mg/kg Cisplatin (greater than 18 and 21 days, respectively) compared to cisplatin administered alone at this dose (10 days).

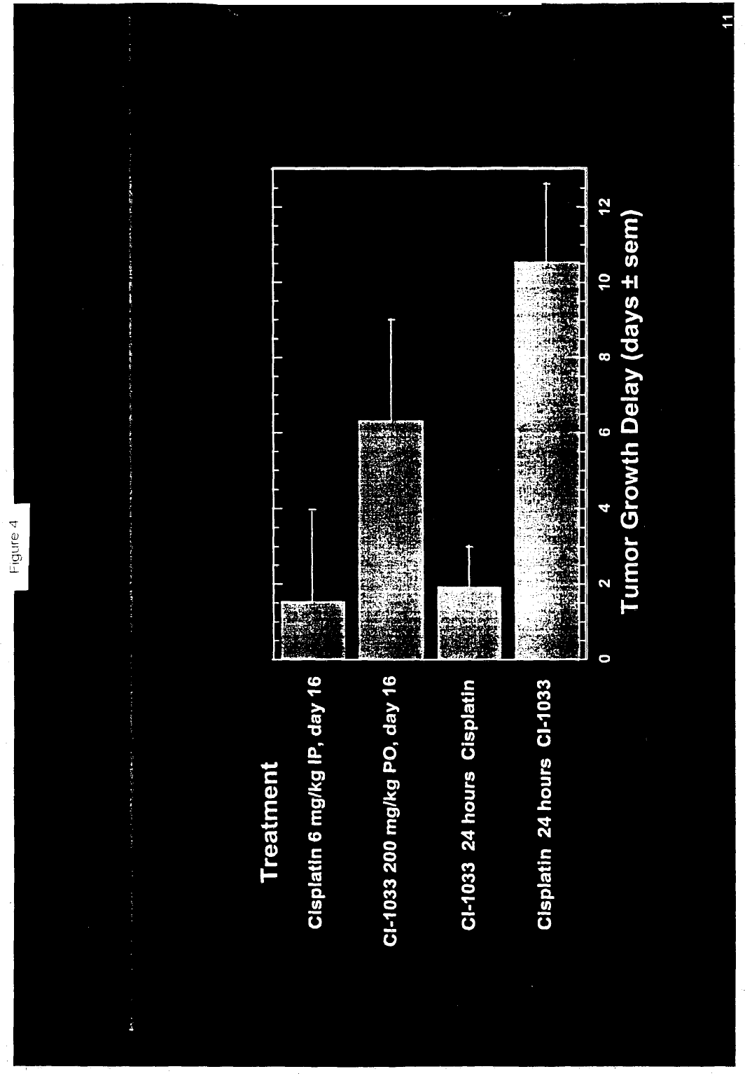

A trial against A431 epidermoid carcinoma was designed to determine tumor sensitivity to cisplatin before and after a single dose of CI-1033. To assess the effect of drug scheduling in therapeutic protocols utilizing CI-1033 and cisplatin, a single dose of 6 mg/kg cisplatin was administered IP to mice bearing advanced A-431 xenografts of Day 16 posttumor implant either 24 hours prior to or after a single dose of either 100 or 200 mg/kg CI-1033. Tumor growth was assessed and the combination of cisplatin followed by CI-1033 produced a greater than additive effect as evidenced by an 11-13.5 day growth delay compared to that produced by cisplatin alone. FIG. 4.

Similar results are obtained using CI-1033 and carboplatin using different dosing schedules.

### EXAMPLE 8

### CI-1033 in Combination with Topotecan

CI-1033 and topotecan were administered to immunodeficient female nude mice having established H125 human cell lung carcinoma xenografts. CI-1033 was administered orally at either 40, 20 or 10 mg/kg on days 32-35 and topotecan was administered IP at doses of 1.6, 1, and 0.62 mg/kg on days 26-30. The combination of CI-1033 and topotecan was evaluated against a H125 human cell lung carcinoma xenograft.

Both CI-1033 and topotecan produced meaningful anticancer effectiveness as measured by tumor growth delay against advanced H125 NSC lung xenografts. Anticancer effectiveness of the combinations were superior to that of either agent administered individually. There was no indication of potentiated toxicity.

Similar results occur with CPT-11, although the treatment schedules may be varied.

### EXAMPLE 9

### CI-1033 in Combination with Radiation Therapy

The synergistic combinations provided by this invention have been evaluated in a murine squamous cell carcinoma, SCC7 implanted subcutaneously in C3H female mice in standard chemotherapy studies using a combination of CI-1033 and ionizing radiation.

Two studies were conducted. In both studies multiple doses of CI-1033 (40, 20, 10 and 5 mg/kg) were administered orally on days 7-18. Radiation was delivered as either a single dose or as multiple fractions over a 5-day period. In these trials, CI-1033 was administered 1 hour before radiation in both the single- and multiple-dose radiation protocols. In the single-dose radiation protocol against SCC-7, the tumors received either 5 or 10 Gy of radiation 1 hour after the first of 12 PO doses of CI-1033 on Day 7. In this trial the SCC-7 carcinoma was insensitive to CI-1033 therapy alone. Single doses of 10 and 5 Gy radiation produced tumor growth delays of 13.6 and 0.8 days, respectively. Combination therapy with radiation plus CI-1033 produced a superior antitumor effect over that obtained with either radiation or CI-1033 therapy alone (91% enhancement). The effect was more pronounced at the 10-Gy radiation dose, with the improved antitumor effect accompanied by an apparent increase in the number of complete and partial regressions.

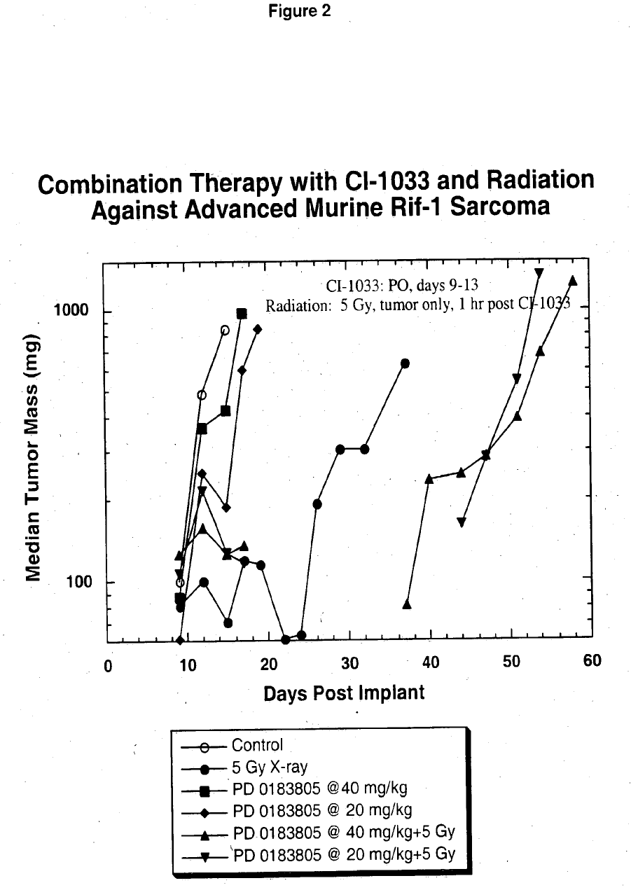

A study combining multiple doses of radiation with multiple doses of CI-1033 was conducted against the Rif-1 sarcoma, based on the effectiveness of the single-dose radiation therapy protocol against SCC-7. This study evaluated the effectiveness of 5 daily doses of radiation administered 1 hour after each of 5 daily doses of CI-1033. As observed against SCC-7, CI-1033 was minimally effective against the Rif-1 sarcoma based on the 3.7-day tumor growth delay produced by the 5-day treatment schedule at the dose used in this study. Radiation at 5 Gy for 5 days produced a tumor growth delay of 28.5 days. Combination therapy with 5 Gy radiation plus CI-1033 produced a surprisingly superior effect compared to that observed with radiation alone (42% enhancement) indicating a superior anticancer effect with this clinically relevant fractionated irradiation schedule. Data representative of this effect is provided on Table I and FIG. 2.

Similar enhancement of CI-1033 in combination with radiation was seen in LoVo tumors, a colon cancer model.

TABLE I
Antitumor Effect of CI-1033 in Combination With X-ray
Against SCC-7 Murine Squamous Cell Carcinoma

| CI-1033 | | X-ray | | Toxic | % Weight | Antitumor Effect | | |
|---|---|---|---|---|---|---|---|---|
| Dose^{a} | Schedule | Dose^{b} | Schedule | Deaths | Change^{c} | CR^{d} | PR^{e} | T − C^{f} |
| 40 | PO^{g}, D 7-18 | | | 0/6 | −2 | | | 0.9 |
| 20 | PO, D 7-18 | | | 0/6 | −1 | | | 0.2 |
| 10 | PO, D 7-18 | | | 0/6 | −1 | | | 1.3 |
| 0 | | 10 | TO^{g}, D 7 | 0/6 | −15 | | | 13.6 |
| 40 | PO, D 7-18 | 10 | TO, D 7 | 0/6 | −14 | 5/6 | | 26.0 |
| 20 | PO, D 7-18 | 10 | TO, D 7 | 0/6 | −15 | 2/6 | 1/6 | 20.4 |
| 10 | PO, D 7-18 | 10 | TO, D 7 | 0/6 | −13 | | 1/6 | 9.7 |
| 5 | PO, D 7-18 | 10 | TO, D 7 | 0/6 | −15 | 2/6 | | 11.1 |

Mice were implanted with 0.2 mL of a 10% tumor brei on Day 0.
Median control tumor mass at first treatment was 75 mg. The study was terminated on Day 44.
^{a}Dose is in mg/kg.
^{b}Dose is in Gray (Gy).
^{c}Maximum treatment-related weight loss, expressed as a percent of initial treatment group weight. A net weight gain is represented by a “+”.
^{d}A complete response represents a tumor that decreased in mass by 100% during the study.
^{e}A partial response represents a tumor that decreased in mass by at least 50% during the study.
^{f}T − C is defined as the difference, in days, for the median treated and control tumors to reach a fixed evaluation size, 750 mg.
^{g}PO, oral therapy; TO, only tumor and adjacent tissues are irradiated, not the entire mouse.

These examples establish an unexpectedly favorable outcome in treating tumors with CI-1033 in combination therapy with a wide variety of antineoplastic chemotherapeutic agents, and with CI-1033 in combination therapy with ionizing radiation. Accordingly, this invention provides a method of treating susceptible neoplasms comprising administering CI-1033 in a therapeutic regimen with one or more other chemotherapeutic agents, pharmaceutically acceptable salts thereof, or ionizing radiation.

The combination of therapeutic agents may be packaged together. The package generally will include each active ingredient packaged separately, thereby avoiding any interaction between the agents prior to administration, as well as individually packaged buffers or diluents for each agent. If desired, the individually packaged drugs can be placed in a single carton as a kit, thereby providing convenience to the attending physician or medical attendant. Such a kit may contain two compartments comprising CI-1033 in one compartment and an antineoplastic agent in a second compartment. A kit having at least three compartments comprising CI-1033 in one compartment and two different antineoplastic agents (together with their separately packaged diluents or buffers, in a second and third compartment, respectively, is also contemplated by this invention.

The susceptible neoplasms to be treated according to this invention include tumors having mutations or over expression of one or more of the erb B receptors. Among the tumors meeting this criterion are solid tumors, especially advanced solid tumors and non-small cell lung cancer, squamous cell carcinoma, glioma, small cell lung carcinoma, endometrial cancer, thyroid cancer, melanoma, colorectal cancer, renal cell cancer, pancreatic cancer, head and neck cancer such as esophageal or cervical cancers, ovarian cancer, myeloma, prostate cancer, sarcomas, chronic myelogenous leukemia and breast cancer.

1. A method of treating cancer comprising administration in a therapeutic regimen of an irreversible panerb B inhibitor and at least one antineoplastic agent selected from the group consisting of gemcitabine, paclitaxel, docetaxel, cisplatin, carboplatin, etoposide, adriamycin, topotecan, CPT-11, capecitabine, and pharmaceutically acceptable salts thereof, wherein said cancer is selected from the group consisting of non-small cell lung cancer, squamous cell carcinoma, glioma, small cell lung carcinoma endometrial cancer, thyroid cancer, melanoma, colorectal cancer, bladder cancer, renal cell cancer, pancreatic cancer, head and neck cancer such as esophageal or cervical cancers, ovarian cancer, myeloma, prostate cancer, sarcomas, chronic myelogenous leukemia and breast cancer.

2. (canceled)

3. (canceled)

4. The method according to claim 1 wherein said panerb B inhibitor is N-[4-(3-chloro-4-fluoro-phenylamino)-7-(3-morpholin-4-yl-propoxy)-quinazolin-6-yl]-acrylamide.

5. (canceled)

6. The method according to claim 1 wherein said at least one antineoplastic agent is gemcitabine or a pharmaceutically acceptable salt thereof.

7. The method according to claim 1 wherein said at least one antineoplastic agent is a taxane or a pharmaceutically acceptable salt thereof.

8. The method according to claim 1 wherein said at least one antineoplastic agent is selected from the group consisting paclitaxel and docetaxel.

9. The method of claim 1, wherein said at least one panerb B inhibitor and said at least one antineoplastic agent is administered in an amount sufficient to inhibit cancer cell growth.

10. (canceled)

11. (canceled)

12. The method of claim 4 comprising administering said panerb B inhibitor in a therapeutic regimen with at least one antineoplastic agent selected from the group consisting of gemcitabine, paclitaxel, docetaxel, cisplatin, carboplatin, etoposide, adriamycin, topotecan, CPT-11, and capecitabine.

13. The method according to claim 1, wherein the antineoplastic agent is administered prior to the panerb B inhibitor.

14. The method according to claim 1, wherein the antineoplastic agent is administered at the same approximate time as the panerb B inhibitor.

15. The method according to claim 1, wherein the antineoplastic agent is administered after the panerb B inhibitor.
